(19) United States
(12) Patent Application Publication
(10) Pub. No.: US 2026/0301945 A1
(43) Pub. Date: Oct. 1, 2026

(54) SYSTEM AND METHOD FOR CLASSIFICATION OF CHRONIC OBSTRUCTIVE PULMONARY DISEASE (COPD)

(71) Applicant: Qure . ai Technologies Private Limited, Mumbai (IN)

(72) Inventors: Bhargava Reddy, Mumbai (IN); Manoj Tadepalli, Mumbai (IN); Anshul Kumer Singh, Mumbai (IN); Rohitashva Agrawal, Mumbai (IN)

(21) Appl. No.: 19/450,721

(22) Filed: Jan. 16, 2026

(30) Foreign Application Priority Data
Feb. 24, 2025 (IN) 202521016052

(51) Int. Cl. G16H 50/20; G06N 20/00; G16H 10/60; G16H 15/00; G16H 30/40; G16H 50/30

(52) U.S. Cl. CPC G16H 50/20; G06N 20/00; G16H 10/60; G16H 15/00; G16H 30/40; G16H 50/30

(57) ABSTRACT

The present subject matter relates to a system (100) and a method (300) for classification of the COPD. The system (100) receives one or more inputs comprising at least one of one or more X-ray images, a clinical information, associated with a user. Further, the system (100) segments one or more lung regions from one or more anatomical structures of the one or more X-ray images. Further, the system (100) identifies one or more regions of interest (ROIs) from the segmented one or more lung regions, and the one or more ROIs are indicative of COPD within the one or more lung regions. Furthermore, the system (100) classifies each of the one or more ROIs into one or more COPD categories, and provides a report indicating the classified one or more COPD categories. Overall, the system (100) provides an efficient and accessible solution for the COPD classification.

### CROSS-REFERENCE TO RELATED APPLICATIONS AND PRIORITY

The present application does claim priority from Indian Patent Application no. 202521016052 filed on 24 Feb. 2025.

### FIELD OF INVENTION

The present invention, in general, relates to the field of image processing and more particularly, relates to a system and a method for classification of chronic obstructive pulmonary disease (COPD).

### BACKGROUND OF THE INVENTION

This section is intended to introduce the reader to various aspects of art, which may be related to various aspects of the present disclosure that are described or claimed below. This discussion is believed to be helpful in providing the reader with background information to facilitate a better understanding of the various aspects of the present disclosure. Accordingly, it should be understood that these statements in this background section are to be read in this light, and not as admissions of prior art. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Conventional AI-based computer-aided diagnosis (CAD) systems for COPD detection primarily rely on medical image analysis, such as X-rays, to identify abnormalities. These models detect visual patterns like diaphragm positioning, air trapping, and hyperinflation, which are indicative of chronic obstructive pulmonary disease (COPD). However, they lack clinical context, making it difficult to differentiate between the COPD subtypes, such as emphysema and chronic bronchitis. Existing AI-powered medical software mainly focuses on detecting emphysema, as it presents more distinct radiographic features, while chronic bronchitis remains harder to classify using image-based methods alone. Due to their exclusive reliance on image data, these systems require large datasets to achieve reliable accuracy, making them computationally expensive. Furthermore, without incorporating patient history, symptoms, and risk factors, these models may risk misclassification or over-reliance on visual cues, which may not fully capture the progression and complexity of the COPD.

Moreover, Computed Tomography (CT) scans are generally used for detailed lung assessments, capturing high-resolution images of lung structures. However, despite their lower resolution being a two-dimensional (2d) image, X-rays remain a valuable source due to their accessibility, affordability, and ability to screen large populations. When combined with clinical information and enhanced by artificial intelligence (AI) techniques, X-rays hold significant potential for chronic obstructive pulmonary disease (COPD) detection. AI compensates for X-ray limitations by identifying subtle disease features, by integrating a contextual clinical information, and enabling early diagnosis, particularly in resource-constrained environments. Further, due to potential advancements in AI algorithms continue to improve the utility of X-rays, making them a viable tool for the widespread COPD screening and management.

However, usage of CT scan based abnormality classification presents significant challenges compared to X-ray-based methods. While CT scans provide highly detailed imaging for capturing subtle structural changes such as emphysema distribution and small airway abnormalities, however usage for the same in abnormality classification comes with notable drawbacks. CT scans are expensive, less accessible due to limited availability, and require specialized equipment and expertise. Due to high radiation exposure for capturing the CT scan and limited frequency of usage, making them unsuitable for regular screening. Additionally, AI-driven CT scan analysis demands extensive datasets and high computational power due to the complexity of image processing. While CT scans are preferred for detailed disease evaluation, severity assessment, and subtype differentiation, their practical limitations hinder widespread application.

On the other hand, Large Language Models (LLMs) have shown strong capabilities in processing unstructured text data, such as clinical notes and patient records. While some LLMs can process image data, their primary strength lies in identifying textual correlations rather than structured clinical insights. For example, an LLM-based CAD system may recognize that smoking history is linked to COPD, but it does not inherently understand medical causation, it merely mimics statistical associations from large datasets. Additionally, LLMs struggle with structured clinical data, which often consists of numerical values and tabular records, making their direct application in diagnostic imaging and classification tasks limited. Thus, conventional AI-based COPD detection systems are either too image-focused i.e., lacking clinical context or too language-driven i.e., struggling with structured medical data. This gap highlights the need for an integrated AI approach that combines deep learning for medical imaging with clinical data processing to enhance COPD classification, risk assessment, and early diagnosis.

Additionally, X-rays can help in identifying structural changes associated with COPD, however due to limited information in 2D X-ray imaging, accurately assessing the severity of disease is challenging. Additionally, in some instances poor-quality X-rays caused by factors like low exposure, insufficient inspiration, or patient misalignment can further hinder reliable classification of the COPD and its associated changes.

In light of the above stated discussion, there exists a need for an improved system and a method for classification of the chronic obstructive pulmonary disease (COPD).

### SUMMARY OF THE INVENTION

Before the present system and device and its components are summarized, it is to be understood that this disclosure is not limited to the system and its arrangement as described, as there can be multiple possible embodiments which are not expressly illustrated in the present disclosure. The present disclosure overcomes one or more shortcomings of the prior art and provides additional advantages discussed throughout the present disclosure. Additional features and advantages are realized through the techniques of the present disclosure. It is also to be understood that the terminology used in the description is for the purpose of describing the versions or embodiments only and is not intended to limit the scope of the present application. This summary is not intended to identify essential features of the claimed subject matter nor is it intended for use in detecting or limiting the scope of the claimed subject matter.

According to embodiments illustrated herein, a method for classification of chronic obstructive pulmonary disease (COPD) is disclosed. In one implementation of the present disclosure, the method may involve various steps performed by a processor. The method may involve a step of receiving one or more inputs. In an embodiment, the one or more inputs may include at least one of one or more X-ray images, a clinical information, or a combination thereof, associated with a user. Further, the method may involve a step of segmenting one or more lung regions from one or more anatomical structures of the one or more X-ray images. Furthermore, the method may involve a step of identifying one or more regions of interest (ROIs) from the segmented one or more lung regions. In an embodiment, the one or more ROIs are indicative of COPD within the one or more lung regions. Furthermore, the method may involve a step of classifying each of the one or more ROIs into one or more COPD categories. Additionally, the method may involve a step of providing a report indicating the one or more COPD categories.

According to embodiments illustrated herein, a system for classification of COPD is disclosed. In one implementation of the present disclosure, the system may involve the processor and a memory. The memory is communicatively coupled to the processor. Further, the memory is configured to store one or more executable instructions. Further, the processor may be configured to receive the one or more inputs. In an embodiment, the one or more inputs may include at least one of the one or more X-ray images, the clinical information, or a combination thereof, associated with the user. Further, the processor may be configured to segment the one or more lung regions from the one or more anatomical structures of the one or more X-ray images. Furthermore, the processor may be configured to identify the one or more regions of interest (ROIs) from the segmented one or more lung regions. In an embodiment, the one or more ROIs are indicative of the COPD within the one or more lung regions. Furthermore, the processor may be configured to classify each of the one or more ROIs into the one or more COPD categories. Additionally, the processor may be configured to provide the report indicating the one or more COPD categories.

According to embodiments illustrated herein, there is provided a non-transitory computer-readable storage medium having stored thereon, a set of computer-executable instructions causing a computer comprising one or more processors to perform various steps. The steps may involve receiving the one or more inputs. In an embodiment, the one or more inputs may include at least one of the one or more X-ray images, the clinical information, or a combination thereof, associated with the user. Further, the steps may involve segmenting the one or more lung regions from the one or more anatomical structures of the one or more X-ray images. Furthermore, the steps may involve identifying the one or more regions of interest (ROIs) from the segmented one or more lung regions. In an embodiment, the one or more ROIs are indicative of the COPD within the one or more lung regions. Furthermore, the steps may involve classifying each of the one or more ROIs into the one or more COPD categories. Additionally, the steps may involve providing the report indicating the one or more COPD categories.

The foregoing summary is illustrative only and is not intended to be in any way limiting. In addition to the illustrative aspects, embodiments, examples, and features described above, further aspects, embodiments, and features will become apparent by reference to the drawings and the following detailed description.

### BRIEF DESCRIPTION OF DRAWINGS

The detailed description is described with reference to the accompanying figures. In the figures, same numbers are used throughout the drawings to refer like features and components. Embodiments of a present disclosure will now be described, with reference to the following diagrams below wherein:

FIG. 1 illustrates a block diagram describing a system (100) for classification of chronic obstructive pulmonary disease (COPD), in accordance with at least one embodiment of present subject matter.

FIG. 2 illustrates a block diagram showing an overview of various components of an application server (101) configured for classification of the COPD, in accordance with at least one embodiment of present subject matter.

FIG. 3 illustrates a flowchart describing a method (300) for classification of the COPD, in accordance with at least one embodiment of present subject matter.

FIG. 4 illustrates an image (400) describing a comprehensive report indicating classification of the COPD in accordance with at least one embodiment of present subject matter, and

FIG. 5 illustrates a block diagram (500) of an exemplary computer system (501) for implementing embodiments consistent with the present subject matter.

It should be noted that the accompanying figures are intended to present illustrations of exemplary embodiments of the present disclosure. These figures are not intended to limit the scope of the present disclosure. It should also be noted that accompanying figures are not necessarily drawn to scale.

### DETAILED DESCRIPTION OF THE INVENTION

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in another embodiments,” “in some embodiments,” “in one embodiment,” or “in an embodiment” in places throughout the specification are not necessarily all referring to the same embodiment. Furthermore, the features, structures or characteristics may be combined in any suitable manner in one or more embodiments.

The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. It must also be noted that, the singular forms “a,” “an,” and “the” include plural references unless the context clearly dictates otherwise. Although any methods similar or equivalent to those described herein can be used in the practice or testing of embodiments of the present disclosure, the exemplary methods are described. The disclosed embodiments are merely exemplary of the disclosure, which may be embodied in various forms.

The terminology “one or more X-ray images” and “X-ray images” has the same meaning and are used alternatively throughout the specification. Further, the terminology “one or more COPD categories” and “COPD categories” has the same meaning and are used alternatively throughout the specification. Furthermore, the terminology “clinical information” and “clinical data” has the same meaning and are used alternatively throughout the specification.

The present disclosure relates to a system for classification of chronic obstructive pulmonary disease (COPD). The system includes a processor and a memory storing instructions that enable the processor to receive one or more inputs. In an embodiment, the one or more inputs may include at least one of one or more X-ray images, a clinical information, or combination thereof, associated with a user. Further, the processor may segment one or more lung regions from one or more anatomical structures of the one or more X-ray images. Furthermore, the processor may identify one or more regions of interest (ROIs) from the segmented one or more lung regions. In an embodiment, the one or more ROIs are indicative of the COPD within the one or more lung regions. Furthermore, the system may classify each of the one or more ROIs into one or more COPD categories. Additionally, the processor may provide a report indicating the one or more COPD categories. This approach improves accuracy and efficiency of the COPD diagnosis and classification by leveraging automated image processing and classification techniques. By integrating the clinical information with the X-ray image data, the system enhances diagnostic precision, aiding healthcare professionals in early detection and treatment planning. Moreover, the report provides a comprehensive assessment, for facilitating informed decision-making and personalized patient care.

To address the limitations of conventional systems for classification of the COPD, the disclosed system utilizes one or more deep learning techniques to analyse the X-ray images while integrating the clinical information to enhance diagnostic accuracy and reliability. Unlike conventional methods that rely on CT scans for detailed lung assessments but face accessibility and cost barriers, the disclosed system provides an efficient alternative for large-scale COPD screening and classification. By segmenting the lung regions, identifying the regions of interest (ROIs), and classifying the COPD severity levels, the system overcomes the inherent challenges of X-ray imaging, such as limited resolution and structural overlap. Additionally, the one or more deep learning techniques compensate for poor-quality X-rays by enhancing image interpretation and extracting subtle disease markers that might be missed by traditional assessment. This approach ensures a cost-effective, scalable, and radiation-efficient solution for early COPD detection, particularly benefiting resource-limited settings where CT scans are impractical for routine screening.

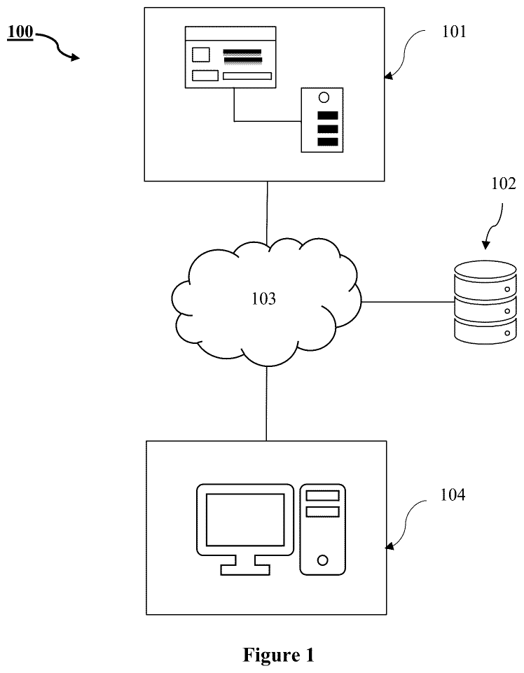

Referring to FIG. 1 is a block diagram that illustrates a system (100) for classification of chronic obstructive pulmonary disease (COPD), in accordance with at least one embodiment of the present subject matter. The system (100) typically comprises an application server (101), a database server (102), a communication network (103), and a user computing device (104). The application server (101), the database server (102), and the user computing device (104) are typically communicatively coupled with each other via the communication network (103). In an embodiment, the application server (101) may communicate with the database server (102), and the user computing device (104) using one or more protocols such as, but not limited to, Hypertext Transfer Protocol (HTTP), Transmission Control Protocol/Internet Protocol (TCP/IP), Wireless Application Protocol (WAP), RF mesh, Bluetooth Low Energy (BLE), and the like, to communicate with one another.

In an embodiment, the database server (102) may refer to a computing device that may be configured to store, manage, and retrieve data associated with the COPD classification system. The database server (102) may include a centralized repository specifically configured for storing and maintaining a structured repository of the one or more X-ray images, the clinical information, segmented lung regions, extracted lung features, identified regions of interest (ROIs), classification results, and corresponding reports. Additionally, the centralized repository may also store parameters relating to the one or more deep learning techniques, training datasets, and historical diagnostic records, facilitating continuous learning and improvement of the automated COPD classification system. The database server (102) enables efficient data retrieval and integration, ensuring seamless processing of a clinical text using one or more language models, and multi-modal techniques for integrating the one or more lung features with clinical embeddings for identifying contextual insights about the COPD. By providing a scalable and secure infrastructure, the database server (102) supports real-time access to COPD classification reports, allowing healthcare professionals to make informed diagnostic and treatment decisions. Moreover, the database server (102) may refer to the computing device configured to perform a variety of database operations essential for classification of the COPD. Further, the centralized repository may be further configured to perform database operations, such as storing, identifying, retrieving, and logging the classification of the COPD into the one or more COPD categories. Moreover, the database server (102) may retrieve. In an exemplary embodiment, logging may correspond to maintaining the clinical information including at least one of user history, risk factor, symptoms, bullae characteristics, typical anatomical changes, age, smoking history, personalized risk profile, clinical guidelines, previous diagnosis notes, family history of lung disease, or a combination of the same. Examples of database operations may include, but are not limited to, storing, retrieving, identifying, managing, and logging data for classification of the chronic obstructive pulmonary disease (COPD). In an embodiment, the database server (102) may include hardware and software capable of being realized through various technologies, such as, but not limited to, Microsoft SQL Server, Oracle, IBM DB2, Microsoft Access, PostgreSQL, MySQL, SQLite, or distributed database technologies. The database server (102) may also be configured to utilize the application server (101) for storage and retrieval of data required for classification of the COPD into the one or more COPD categories.

A person with ordinary skills in art will understand that the scope of the disclosure is not limited to the database server (102) as a separate entity. In an embodiment, the functionalities of the database server (102) can be integrated into the application server (101) or into the user computing device (104).

In an embodiment, the application server (101) may refer to a computing device or a software framework hosting an application or a software service. In an embodiment, the application server (101) may be implemented to execute procedures such as, but not limited to, programs, routines, or scripts stored in the database server (102) for supporting the hosted application or the software service. In an embodiment, the hosted application or the software service may be configured to perform one or more predetermined operations. The application server (101) may be realized through various types of application servers such as, but are not limited to, a Java application server, a . NET framework application server, a Base4 application server, a PHP framework application server, or any other application server framework.

In an embodiment, the application server (101) may be configured to utilize the database server (102) and the user computing device (104) in conjunction for classification of the COPD. In an implementation, the application server (101) corresponds to a computing system that facilitates the coordination and processing of data between the database server (102) and the user computing device (104). The application server (101) manages the flow of data by retrieving relevant clinical information, the X-ray images, and the classification results from the database server (102), and then communicates with the user computing device (104) to present the processed results to the user or the healthcare professional. The application server (101) also hosts the necessary software applications and deep learning techniques for automated analysis, classifying regions of interest (ROIs), and providing reports. By acting as an intermediary between the database server (102) and user interfaces, the application server (101) ensures smooth data integration, efficient processing, and accurate COPD classification to support clinical decision-making Further, by leveraging the one or more language models to process the clinical information and encode the clinical text into the clinical embeddings, and by integrating the clinical embeddings with the lung features to provide contextual insights, the application server (101), coupled with the database server (102), ensures the accurate processing of the clinical information, such as patient history, symptoms, and risk factors.

In an embodiment, the application server (101) may be configured to receive the one or more inputs. In an embodiment, the one or more inputs may include at least one of the one or more X-ray images, the clinical information, or a combination thereof, associated with the user.

In an embodiment, the application server (101) may be configured to segment the one or more lung regions from the one or more anatomical structures of the one or more X-ray images.

In an embodiment, the application server (101) may be configured to identify the one or more regions of interest (ROIs) from the segmented one or more lung regions. In an embodiment, the one or more ROIs may be indicative of the COPD within the one or more lung regions.

In an embodiment, the application server (101) may be configured to classify each of the one or more ROIs into the one or more COPD categories.

In an embodiment, the application server (101) may be configured to provide the report indicating the one or more COPD categories, to the user.

In an embodiment, the communication network (103) may correspond to a communication medium through which the application server (101), the database server (102), and the user computing device (104) may communicate with each other. Such communication may be performed in accordance with various wired and wireless communication protocols. Examples of such wired and wireless communication protocols include, but are not limited to, Transmission Control Protocol and Internet Protocol (TCP/IP), User Datagram Protocol (UDP), Hypertext Transfer Protocol (HTTP), Wireless Application Protocol (WAP), File Transfer Protocol (FTP), ZigBee, EDGE, infrared IR), IEEE 802.11, 802.16, 2G, 3G, 4G, 5G, 6G, 7G cellular communication protocols, and/or Bluetooth (BT) communication protocols. The communication network (103) may either be a dedicated network or a shared network. Further, the communication network (103) may include a variety of network devices, including routers, bridges, servers, computing devices, storage devices, and the like. The communication network (103) may include, but is not limited to, the Internet, intranet, a cloud network, a Wireless Fidelity (Wi-Fi) network, a Wireless Local Area Network (WLAN), a Local Area Network (LAN), a cable network, the wireless network, a telephone network (e.g., Analog, Digital, POTS, PSTN, ISDN, xDSL), a telephone line (POTS), a Metropolitan Area Network (MAN), an electronic positioning network, an X.25 network, an optical network (e.g., PON), a satellite network (e.g., VSAT), a packet-switched network, a circuit-switched network, a public network, a private network, and/or other wired or wireless communications network configured to carry data.

In an embodiment, the user computing device may comprise one or more processors and one or more memory. The one or more memory may store computer-readable instructions that are executable by the one or more processors to interact with the database server (102) and the application server (101) for efficient classification of the COPD. These instructions enable the computing device to receive and display the processed results from the application server (101), including X-ray images, clinical information, and COPD classification report. The user computing device facilitates the presentation of the processed data, including the clinical information, the X-ray images, and the classification results, to the user or the healthcare professional. The user computing device allows for seamless communication with the application server (101) to receive the classification reports, enabling users to review and interpret the results. Additionally, the user computing device supports the execution of various user interface applications, making it easier to access, visualize, and understand the COPD-related insights provided by the system. Through this integration, the user computing device plays an important role in ensuring that healthcare professionals are able to make well-informed decisions based on real-time monitoring of the input x-ray images along with the clinical information using one or more deep learning techniques.

The system (100) can be implemented using hardware, software, or a combination of both, which includes using where suitable, one or more computer programs, mobile applications, or “apps” by deploying either on-premises over the corresponding computing terminals or virtually over cloud infrastructure. The system (100) may include various micro-services or groups of independent computer programs which can act independently in collaboration with other micro-services. The system (100) may also interact with a third-party or external computer system. Internally, the system (100) may be the central processor of all requests for transactions by the various actors or users of the system. A critical attribute of the system (100) is that the system seamlessly integrates an analysis of the one or more X-ray images with the clinical information to provide accurate, real-time classification of the chronic obstructive pulmonary disease (COPD). By combining the X-ray images, the clinical information, and advanced deep learning techniques, the system enhances diagnostic precision and enables early detection of the COPD, even in resource-limited environments. The system's ability to generate comprehensive reports, segment lung regions, identify regions of interest (ROIs), and classify the COPD into distinct categories ensures a holistic approach to patient care. This integration of X-ray imaging and the clinical information, coupled with automated analysis, enables healthcare professionals to make well-informed decisions, improving the overall efficiency and effectiveness of the COPD classification.

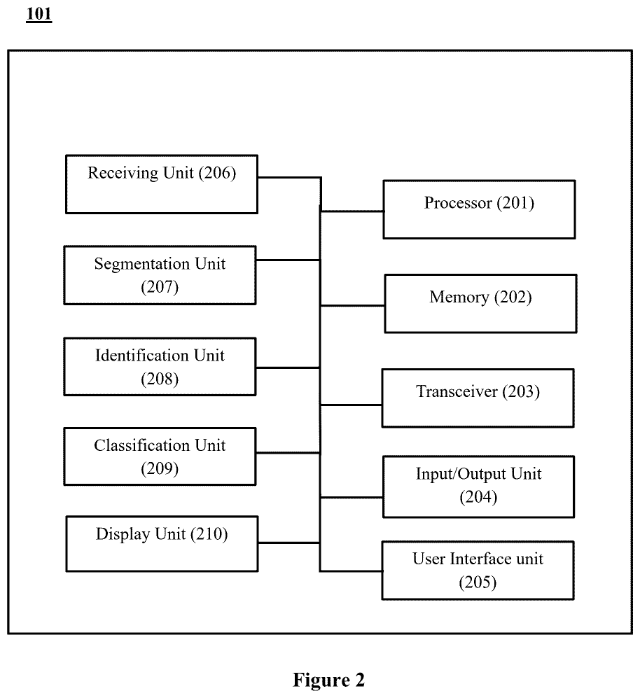

Now referring to FIG. 2, illustrates a block diagram showing an overview of various components of the application server (101) configured for classification of the COPD, in accordance with at least one embodiment of the present subject matter. FIG. 2 is explained in conjunction with elements from FIG. 1. In an embodiment, the application server (101) includes a processor (201), a memory (202), a transceiver (203), an input/output unit (204), a user interface unit (205), a receiving unit (206), a segmentation unit (207), an identification unit (208), a classification unit (209), and a display unit (210). The processor (201) may be communicatively coupled to the memory (202), the transceiver (203), the input/output unit (204), the user interface unit (205), the receiving unit (206), the segmentation unit (207), the identification unit (208), the classification unit (209), and the display unit (210). The transceiver (203) may be communicatively coupled to the communication network (103) of the system (100).

In an embodiment, the system (100) provides a comprehensive solution for the classification of the chronic obstructive pulmonary disease (COPD) by integrating advanced artificial intelligence (AI) techniques with medical imaging and clinical data. The system (100) processes the X-ray images and the clinical information to accurately segment the lung regions, identify the regions of interest (ROIs), and classify the ROIs into specific COPD categories. The system (100) further provides a detailed report to healthcare professionals, offering insights into the severity of the disease and personalized risk scores. Through its automated analysis, the system (100) enables early detection, allowing for timely intervention and improved patient outcomes. The seamless integration of clinical imaging and clinical data, coupled with real-time processing capabilities, ensures that healthcare providers are able to make informed decisions based on accurate, data-driven insights, thereby optimizing the COPD classification.

The processor (201) comprises suitable logic, circuitry, interfaces, and/or code that may be configured to execute a set of instructions stored in the memory (202), and may be implemented based on several processor technologies known in the art. The processor (201) works in coordination with the memory (202), the transceiver (203), the input/output unit (204), the user interface unit (205), the receiving unit (206), the segmentation unit (207), the identification unit (208), the classification unit (209), and the display unit (210) for classification of the COPD. Examples of the processor (201) include, but not limited to, a standard microprocessor, microcontroller, central processing unit (CPU), an X86-based processor, a Reduced Instruction Set Computing (RISC) processor, an Application-Specific Integrated Circuit (ASIC) processor, and a Complex Instruction Set Computing (CISC) processor, distributed or cloud processing unit, state machines, logic circuitries, and/or any devices that manipulate signals based on operational instructions and/or other processing logic that accommodates the requirements of the present invention.

The memory (202) comprises suitable logic, circuitry, interfaces, and/or code that may be configured to store the set of instructions, which are executed by the processor (201). Preferably, the memory (202) is configured to store one or more programs, routines, or scripts that are executed in coordination with the processor (201). Additionally, the memory (202) may include any computer-readable medium or computer program product known in the art including, for example, volatile memory, such as static random-access memory (SRAM) and dynamic random-access memory (DRAM), and/or non-volatile memory, such as read-only memory (ROM), erasable programmable ROM, a Hard Disk Drive (HDD), flash memories, Secure Digital (SD) card, Solid State Disks (SSD), optical disks, magnetic tapes, memory cards, virtual memory and distributed cloud storage. The memory (202) may be removable, non-removable, or a combination thereof. Further, the memory (202) may include routines, programs, objects, components, data structures, etc., which perform particular tasks or implement particular abstract data types. The memory (202) may include programs or coded instructions that supplement the applications and functions of the system (100). In one embodiment, the memory (202), amongst other things, serves as a repository for storing data processed, received, and generated by one or more of the programs or the coded instructions. In yet another embodiment, the memory (202) may be managed under a federated structure that enables the adaptability and responsiveness of the application server (101).

The transceiver (203) comprises suitable logic, circuitry, interfaces, and/or code that may be configured to receive, process or transmit information, data or signals, which are stored by the memory (202) and executed by the processor (201). The transceiver (203) is preferably configured to receive, process or transmit, one or more programs, routines, or scripts that are executed in coordination with the processor (201). The transceiver (203) is preferably communicatively coupled to the communication network (103) of the system (100) for communicating all the information, data, signals, programs, routines or scripts through the communication network (103).

The transceiver (203) may implement one or more known technologies to support wired or wireless communication with the communication network (103). In an embodiment, the transceiver (203) may include but is not limited to, an antenna, a radio frequency (RF) transceiver, one or more amplifiers, a tuner, one or more oscillators, a digital signal processor, a Universal Serial Bus (USB) device, a coder-decoder (CODEC) chipset, a subscriber identity module (SIM) card, and/or a local buffer. Also, the transceiver (203) may communicate via wireless communication with networks, such as the Internet, an Intranet and/or a wireless network, such as a cellular telephone network, a wireless local area network (LAN) and/or a metropolitan area network (MAN). Accordingly, the wireless communication may use any of a plurality of communication standards, protocols and technologies, such as: Global System for Mobile Communications (GSM), Enhanced Data GSM Environment (EDGE), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email, instant messaging, and/or Short Message Service (SMS).

The input/output (I/O) unit (204) comprises suitable logic, circuitry, interfaces, and/or code that may be configured to receive or present information. The input/output unit (204) comprises various input and output devices that are configured to communicate with the processor (201). Examples of the input devices include but are not limited to, a keyboard, a mouse, a joystick, a touch screen, a microphone, a camera, and/or a docking station. Examples of the output devices include, but are not limited to, a display screen and/or a speaker. The I/O unit (204) may include a variety of software and hardware interfaces, for example, a web interface, a graphical user interface, and the like. The I/O unit (204) may allow the system (100) to interact with the user directly or through the user computing devices (104). Further, the I/O unit (204) may enable the system (100) to communicate with other computing devices, such as web servers and external data servers (not shown). The I/O unit (204) can facilitate multiple communications within a wide variety of networks and protocol types, including wired networks, for example, LAN, cable, etc., and wireless networks, such as WLAN, cellular, or satellite. The I/O unit (204) may include one or more ports for connecting a number of devices to one another or to another server. In one embodiment, the I/O unit (204) allows the application server (101) to be logically coupled to other user computing devices (104), some of which may be built in. Illustrative components include tablets, mobile phones, wireless devices, etc.

Further, the input/output (I/O) unit (204) may be configured to manage the exchange of data between the application server (101) and the user computing device (104), ensuring that the data is smoothly communicated between the user and the COPD classification system. The I/O unit (204) handles the transmission of the clinical information, the X-ray images, and the classification results, allowing the user to access real-time reports and contextual insights on the COPD classification. In an exemplary embodiment, the I/O unit (204) ensures seamless communication by managing data encoding, decoding, and error-checking, and guarantees that the data is delivered in a timely and accurate manner. By enabling reliable and efficient data transfer, the I/O unit (204) optimizes the user experience and supporting the system's overall functionality, enabling healthcare professionals to make quick and informed decisions based on the processed COPD data.

Further, the user interface unit (205) may facilitate interaction between the user and the system (100) by providing the report indicating the one or more COPD categories, to the user. The user interface unit (205) enables the user or the healthcare professional to easily input the x-ray images or the clinical information, view the processed X-ray images, and interpret the COPD classification results. The interface may display detailed report, including the identified regions of interest (ROIs), classification categories, and confidence scores, in a user-friendly format. In an exemplary embodiment, the user interface unit (205) may allow the user to interact with the data by enabling the user to zoom in on specific lung regions, compare different X-ray images, and adjust settings for further analysis. The user interface unit (205) may also facilitate the viewing of additional contextual information such as personalized risk scores, family history, symptoms, and other clinical details that are relevant to the classification results. This integration of clinical data and diagnostic insights helps the user better understand the severity of COPD. By offering an accessible and streamlined experience, the user interface unit (205) ensures that the user can efficiently interact with the system, enabling quick, informed decisions in the diagnostic and treatment process.

Further, the receiving unit (206) may be configured to receive the one or more inputs. In an embodiment, the one or more inputs may include at least one of the one or more X-ray images, the clinical information, associated with the user. In an exemplary embodiment, the clinical information may include at least one of user history, risk factor, symptoms, bullae characteristics, typical anatomical changes, age, smoking history, personalized risk profile, clinical guidelines, previous diagnosis notes, family history of lung disease, or a combination of the same.

Furthermore, the receiving unit (206) may be configured to preprocess the one or more X-ray images. In an embodiment, the preprocessing may correspond to one of noise reduction, contrast adjustment, normalization, or a combination of the same. In an embodiment, the preprocessing of the one or more X-ray images may be performed before segmenting the one or more lung regions from the one or more X-ray images.

Furthermore, the segmentation unit (207) may be configured to segment the one or more lung regions from the one or more anatomical structures of the one or more X-ray images. In an exemplary embodiment, segmenting of the one or more lung regions from one or more anatomical structures may be performed using one or more deep learning models. In an embodiment, the one or more deep learning models may correspond to one of a UNet or a UNet++, DeepLab, Fully Convolutional Network (FCN), SegNet, R2U-Net (Recurrent Residual U-Net), ResUNet (U-Net with ResNet backbone), Densely Connected U-Net (DC-UNet), Mask R-CNN, or a combination of the same.

In one embodiment, the processor (201) may be configured for training the one or more deep learning models by utilizing the one or more X-ray images to identify one or more pixels, one or more features, one or more patterns associated with the one or more anatomical structures. In a related embodiment, the segmentation unit (207) may be configured for segmenting the one or more lung regions from the one or more anatomical structures is performed based on the one or more pixels, one or more features, and one or more patterns identified during the training.

Furthermore, the identification unit (208) may be configured to identify the one or more regions of interest (ROIs) from the segmented one or more lung regions. In an embodiment, the one or more ROIs may be indicative of the COPD within the one or more lung regions. In one embodiment, identifying the one or more regions of interest (ROIs) may be performed by processing the segmented one or more lung regions utilizing at least one of a region-based convolutional neural network (R-CNN) deep learning model, a transformer-based neural network model, or a combination of the same. The processing of the segmented one or more lung regions may comprise extracting the one or more lung features from the segmented one or more lung regions. In an embodiment, each of the one or more lung features may be associated with a weightage. Furthermore, the identification unit (208) may be configured to process the segmented one or more lung regions by analyzing one or more patterns from the one or more lung features. Furthermore, the identification unit (208) may be configured to process the segmented one or more lung regions by integrating the one or more lung features and the one or more patterns, indicative of the COPD, for identifying the one or more ROIs. In an embodiment, each of the one or more ROIs may be assigned with a classification score based on the weightage associated with the one or more features.

In another embodiment, processing the segmented one or more lung regions may be performed at different scales to extract one or more lung features and analyze one or more patterns, indicative of the COPD. In an exemplary embodiment, the one or more lung features may include low-level features and high-level features. In an embodiment, the low-level features comprise at least one of edges, gradients, shapes, texture or a combination of the same. In an embodiment, the high-level features may include at least one of thickness, curvature, relative position, variation in pixel intensity, or a combination of the same. In another exemplary embodiment, the one or more patterns may include at least one of flattened diaphragm, thickened bronchial walls, hyperinflation, bronchial thickening, radiolucency, position of diaphragm, narrow heart, air bullae in upper lobes, edges of the diaphragm, edges of bronchial wall, texture and thickness of bronchial walls, hallmark of hyperinflated lungs.

Additionally, the identification unit (208) identifies critical COPD-related patterns, including flattened diaphragms, thickened bronchial walls, hyperinflation, radiolucency, abnormal diaphragm positioning, narrow heart, air bullae in upper lobes, and texture variations in bronchial walls, which serve as key indicators of disease presence and severity. By integrating these feature hierarchies with AI-driven analysis, the identification unit (208) enhances COPD detection, classification, and clinical decision-making, providing a robust, automated tool for respiratory disease assessment.

In an exemplary embodiment, a Convolution Neural Network (CNN) may identify the ROIs in the lung region by processing chest X-ray images through multiple convolutional layers. Early layers may detect the low-level features such as edges and textures, while deeper layers combine these features to recognize higher-level features like shapes and structures. For identifying the COPD, the one or more region of interests (ROIs) may include regions showing signs of hyperinflation, flattened diaphragms, or reduced vascular markings. By learning these hierarchical feature representations, the CNN discerns regions indicative of abnormalities and uses this information to make accurate predictions.

In an exemplary embodiment, in CNN-based medical image analysis, the one or more lung features are measurable visual elements like edges, textures, or shapes extracted from images, while the one or more patterns are broader recurring structures or arrangements, such as flattened diaphragms or hyperinflation indicative of the COPD. Herein, the one or more features may serve as the building blocks that the CNN combines to recognize and interpret the one or more patterns, enabling accurate detection and classification of abnormalities in chest X-rays. An artificial intelligence (AI) model in its earlier layer extracts the low-level features like edges and curves and combines the low-level features in the later layer to identify the shape and structures like flattened diaphragm for Emphysema.

In an exemplary embodiment, during the training process, the AI model is provided with a large dataset of X-ray images accompanied by expert-annotated lung regions. These annotations serve as a reference to guide the AI model in recognizing visual patterns and accurately identifying regions of interest. Through multiple iterations, the AI model progressively improves the ability to segment the target regions by receiving feedback and penalties for incorrect predictions. This iterative refinement ensures that the AI model focuses exclusively on the relevant lung structures, minimizing the influence of surrounding anatomical features. Furthermore, the process involves training the one or more deep learning models by utilizing the one or more X-ray images to identify one or more pixels, the one or more features, the one or more patterns associated with the one or more anatomical structures. In an embodiment, segmenting the one or more lung regions from the one or more anatomical structures may be performed based on the one or more pixels, the one or more features, and the one or more patterns identified during the training.

In an exemplary embodiment, the AI model does not use manually provided rules to integrate the one or more features from the clinical information. The AI model integrates features from the one or more X-ray images and the clinical information using multimodal neural networks. The vision component extracts visual features indicative of COPD, such as flattened diaphragms or hyperinflation, while the language component processes clinical data like symptoms, patient history, or radiology notes. These features are fused in a shared feature space, enabling the AI to align imaging findings with clinical context. For example, the X-ray image showing hyperinflation combined with clinical notes of smoking history and chronic cough would result in a higher likelihood of COPD. This integration improves diagnostic accuracy by providing context-aware predictions

In yet another exemplary embodiment, the CNN doesn't explicitly label the one or more lung features as low-level or high-level. Instead, these terms describe the natural progression of feature learning within the convolutional neural network. For example, the early layers of the CNN capture simple, low-level features such as edges, textures, or basic shapes. As the data passes through subsequent layers, these features are progressively combined and transformed into complex, high-level representations that encapsulate abstract patterns relevant to the COPD classification. This hierarchical learning process is analogous to constructing an object from fundamental building blocks, gradually assembling them into a complete structure. The AI model's final output is derived from this layered composition of features, meaning that the accuracy of individual low-level features or high-level features in isolation is less significant than their collective contribution to the final classification. The true effectiveness of the CNN lies in how well these features interact to generate a precise and reliable COPD diagnosis.

In an exemplary embodiment, the identification unit (208) employs a hierarchical feature extraction framework, distinguishing between low-level and high-level lung features to enhance COPD classification accuracy. At the low level, the system captures fundamental structural attributes, including edges and boundaries, which detect sharp intensity transitions outlining anatomical structures such as clear lung field borders and distinct diaphragm edges. The identification unit (208) also analyzes texture patterns, identifying fine pixel variations such as distinguishing granular texture in normal lung tissue from irregular patches in affected areas. The identification unit (208) further measures intensity gradients, evaluating local brightness shifts indicative of tissue density changes, such as gradual transitions suggesting consolidation or abrupt changes at air-tissue interfaces. Additionally, the identification unit (208) further measures local contrast variations enable differentiation of subtle contrast differences within localized regions, helping to distinguish healthy parenchyma from focal infiltrates.

At the high level, the identification unit (208) synthesizes these low-level features into more complex diagnostic patterns. The identification unit (208) evaluates global anatomical structures, assessing overall lung morphology, spatial relationships, and lung field symmetry relative to the heart and diaphragm. The identification unit (208) identifies emphysema radiographic patterns, detecting upper lobe hyperlucency, bullae formation, and reduced vascular markings indicative of alveolar destruction. For chronic bronchitis, the identification unit (208) integrates features such as mild hyperinflation, increased bronchial markings, and bronchial wall thickening, which are indicative of airway inflammation. The identification unit (208) further analyzes regional abnormalities and spatial relationships, assessing the distribution and extent of abnormal density changes, such as diffuse patchy opacities or localized consolidations. Finally, the identification unit (208) synthesizes integrated disease signatures, combining multiple diagnostic cues into a coherent classification, such as hyperlucency with sparse vasculature for emphysema or bronchial thickening with hyperinflation for chronic bronchitis.

In an exemplary embodiment, the identification unit (208) may be configured to process the clinical information using the one or more language models. In an embodiment, processing the clinical information may include parsing a clinical text from the clinical information and encoding the clinical text into the clinical embeddings. In an embodiment, the one or more language models may correspond to NLP, LLM, or a combination of the same.

In an exemplary embodiment, the identification unit (208) may be configured to integrate the one or more lung features with the clinical embeddings for identifying contextual insights about the COPD. In an embodiment, integration may be performed using one or more multi-modal techniques. In an exemplary embodiment, the one or more multi-modal techniques may include one of co-attention mechanism, cross attention mechanism, late fusion, or a combination of the same.

In an exemplary embodiment, a transformer-based co-attention module may be used to integrate the one or more features from both image and text modalities, creating a unified representation that captures relevant information from each source. This fused representation may be then passed through a classification head, typically a feed-forward network, which outputs probability scores for predefined categories. By effectively combining information from both modalities, the co-attention module enhances the classification model's ability to make accurate and reliable predictions.

In yet another exemplary embodiment, a rule-based logic may be used as well to generate the final probability score for three abnormalities. A vision model may generate the probability score which may be modified based on the information present in the clinical information like symptoms, patient history, risk factors, or a combination of the same. The probability may be updated using the Bayes'rule.

$$P ( Abnormality \vert Evidence ) = P ( Evidence \vert Abnormality ) * P ( Abnormality ) / P ( Evidence )$$

Wherein P(Evidence) corresponds to probability of observing an evidence.

Wherein P(Evidence|Abnormality) corresponds to probability of observing the evidence when the abnormality is present.

Wherein P(Abnormality|Evidence) corresponds to probability of the abnormality to be present when the evidence is observed.

Further, the classification unit (209) may be configured to classify each of the one or more ROIs into the one or more COPD categories.

In an exemplary embodiment, the classifying each of the one or more ROIs into the one or more COPD categories may be performed based on the one or more lung features, the one or more patterns, the clinical embeddings, or a combination of the same. In an exemplary embodiment, the one or more COPD categories may include None, Emphysema and Chronic Bronchitis. The None as the COPD category indicates that there is no possibility of having COPD, thus no immediate concern. Further, Emphysema as the COPD category indicates that a detailed investigation is required for the confirmation of emphysema in the user. Further, Chronic Bronchitis as the COPD category indicates that a detailed investigation is required for the confirmation of Chronic Bronchitis in the user. In an exemplary embodiment, classifying each of the one or more ROIs into the one or more COPD categories may be performed by utilizing at least one of logistic regression, Support Vector Machine (SVM), feed forward network (MLP), transformer-based classifier, or a combination of the same. In an embodiment, each of the one or more COPD categories may be assigned with a confidence score. In an embodiment, a cumulative sum of each of the confidence score corresponding to the one or more COPD categories is equal to one.

In an embodiment, classification of a ROI from the one or more ROIs may correspond to a COPD category from the one or more COPD categories, with a highest confidence score among other COPD categories. For example, if the classification model assigns a higher confidence score to the Emphysema category based on the analysis of the lung features and the patterns, the system will classify that particular ROI as indicative of Emphysema. Similarly, if another ROI shows the higher confidence score for Chronic Bronchitis, the system will classify that particular ROI as indicative of Chronic Bronchitis. This approach ensures that the classification reflects the most probable COPD category based on the classification model's analysis, enabling the user or the healthcare professionals to receive accurate and reliable diagnostic information for further clinical decision-making. In an exemplary embodiment, the confidence score indicates the level of certainty of the classification model, helping to guide the interpretation of the results.

In an exemplary embodiment, the classification unit (209) classifies COPD into None, Emphysema, or Chronic Bronchitis by learning to detect features commonly observed in these conditions. For Emphysema, the classification unit (209) identifies patterns such as hyperinflation/flattened diaphragms, reduced vascular markings, and bullae. For Chronic Bronchitis, the classification unit (209) detects bronchial wall thickening, increased bronchovascular markings, and air trapping. Clinical data, such as smoking history, dyspnea, or chronic productive cough, are also incorporated to provide contextual information. During inference, the classification unit (209) assigns confidence scores to each category based on these detected features, with the highest score determining the final classification.

In an exemplary embodiment, an artificial intelligence (AI) based technique in detecting Emphysema and Chronic Bronchitis focuses on visually justifying the predictions by highlighting suspicious areas on X-rays. For example, in Emphysema detection, the AI-based techniques highlight regions of hyperinflation, such as flattened diaphragms or reduced vascular markings, as reasons for its prediction. Similarly, for Chronic Bronchitis, the AI-based techniques highlight areas like bronchial wall thickening and increased bronchovascular markings. Along with the prediction, the AI-based techniques provide these highlighted regions to show the basis of the decision, ensuring transparency and aiding clinicians in understanding and verifying the diagnosis.

In another embodiment, the classification unit (209) may be configured to calculate a personalized risk score, corresponding to the user, based on the one or more COPD categories and the risk factor associated with the user. In an embodiment, the personalized risk score may be indicative of an estimate of likelihood of the COPD. For example, if the user has a history of smoking, a family history of lung disease, and shows early signs of Emphysema, the classification model may assign a higher risk score, indicating a greater likelihood of the COPD progression. This personalized risk score may assist healthcare professionals in assessing the user's current condition and determining an appropriate course of action, including preventive measures or more intensive monitoring.

In an exemplary embodiment, the classification unit (209) may generate a normalized score, derived from the user's X-ray image and the clinical information, to create a personalized risk assessment specific to the individual. This score reflects the user's unique health profile by integrating both imaging data and clinical risk factors, such as smoking history, family history of lung disease, and early signs of emphysema. In this context, the normalized score is equivalent to the personalized risk score, offering a comprehensive and individualized evaluation of the COPD likelihood. A higher risk score may indicate greater disease progression, prompting closer monitoring or early intervention, while a lower risk score suggests a minimal risk of the COPD development. By combining deep learning techniques with personalized data analysis, the system enhances clinical decision-making.

Furthermore, the display unit (210) may be configured to provide the report indicating the one or more COPD categories.

In an exemplary embodiment, the one or more COPD categories may be provided over the one or more X-ray images in the report. In an embodiment, the report may include the one or more COPD categories, personalized risk score and an explanation of findings. For example, if the analysis indicates Emphysema with a high confidence score, the report may display the affected lung regions with a clear label and provide a detailed explanation of how the features, such as hyperinflation or radiolucency, support this classification. Additionally, the personalized risk score would offer insights into the likelihood of disease progression, helping the healthcare professional make informed decisions regarding the patient's care and treatment options. In an embodiment, various components of the application server (101) may utilize explainable AI (XAI) to justify various findings made by the components. The justification by the XAI is provided as a part of the report in such a way, which is easily interpretable by healthcare professionals.

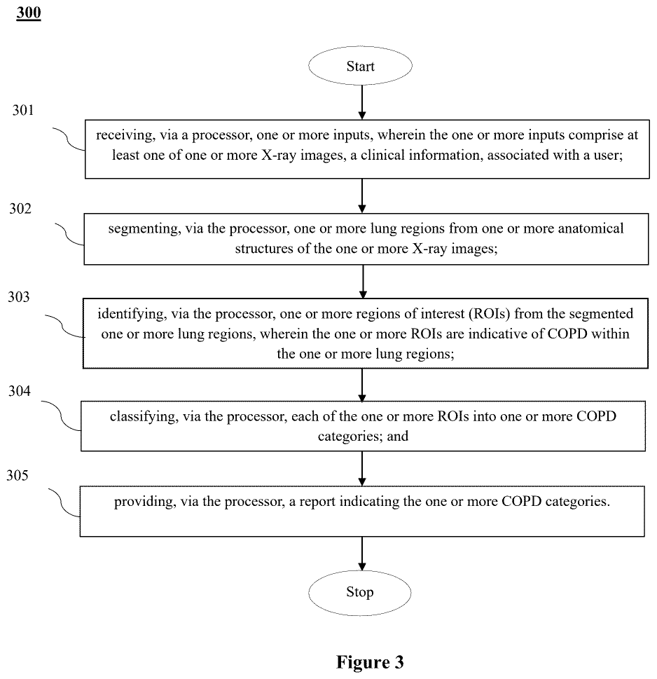

Now referring to FIG. 3, illustrates a flowchart describing a method (300) for classification of the COPD, in accordance with at least one embodiment of the present subject matter. The flowchart is described in conjunction with FIG. 1 and FIG. 2. The method (300) starts at step (301) and proceeds to step (305).

In operation, the method (300) may involve a variety of steps, executed by the processor (201), for classification of the COPD.

At step (301), the method involves receiving the one or more inputs. In an embodiment, the one or more inputs may include at least one of the one or more X-ray images, the clinical information, associated with the user.

At step (302), the method involves segmenting the one or more lung regions from the one or more anatomical structures of the one or more X-ray images.

At step (303), the method involves identifying the one or more regions of interest (ROIs) from the segmented one or more lung regions. In an embodiment, the one or more ROIs may be indicative of the COPD within the one or more lung regions.

At step (304), the method involves classifying each of the one or more ROIs into the one or more COPD categories.

At step (305), the method involves providing the report indicating the one or more COPD categories, to the user.

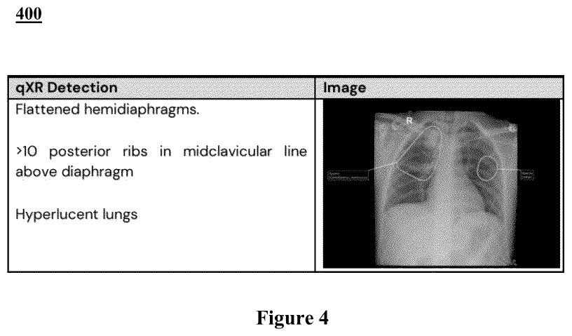

Now referring to FIG. 4 illustrates an image (400) describing a comprehensive report indicating classification of the COPD in accordance with at least one embodiment of present subject matter. In an exemplary embodiment, a right section of the image (400) displays an annotated chest X-ray image highlighting the specific regions of interest (ROIs) corresponding to the one or more lung features identified by the system (100). The ROIs are outlined and associated with the one or more patterns, indicative of potential abnormalities. Furthermore, a left section of the image (400) presents findings of the annotated chest X-ray image (i. e qXR Detection) in a textual format, describing the one or more patterns associated with the identified one or more lung features, indicative of the COPD. The one or more patterns include flattened hemidiaphragms, hyperlucent lungs, and the observation of more than 10 posterior ribs in the midclavicular line above the diaphragm. Such findings suggest hyperinflation, a hallmark feature often associated with the COPD. Thus, by integrating the clinical information with the X-ray image data, the system (100) enhances diagnostic precision, aiding healthcare professionals in early detection and treatment planning. Moreover, the system (100) generates the comprehensive report detailing the identified COPD classification, thereby facilitating informed decision-making and personalized patient care.

### Working Example 1

Consider a scenario for identifying a high-risk bullae for COPD Classification. A 5 cm bullae is detected in the right upper lobe of a patient's lung. Bullae, which are air-filled spaces in the lungs, are known risk factors for COPD, particularly when they exceed a certain size threshold. In this case, the large size of the bullae increases the likelihood of COPD progression, making it a clinically significant finding.

From a machine learning perspective, this example serves as an objective indicator for COPD detection. The CAD model learns to prioritize larger bullae as high-risk features during training. By associating size and location with increased disease severity, the model enhances its ability to distinguish between normal anatomical variations and pathological bullae that warrant further clinical evaluation. This training process improves the AI's capability to detect and assess COPD severity, allowing it to provide more accurate risk assessments in real-world scenarios.

### Working Example 2

Consider a scenario where a clinician initiates the process by uploading the patient's X-ray and entering essential clinical details, such as the patient's age, smoking history, and family history of lung disease. The system then performs preprocessing on the X-ray image, applying noise reduction and contrast adjustment techniques to enhance the image quality and standardize the data, ensuring it is suitable for AI analysis.

The next step involves image analysis, where the system segments the lung ROIs, particularly focusing on areas where bullae, which are air-filled spaces in the lungs, might be present. Using a deep learning algorithm, the system detects and isolates potential bullae, analyzing their size, shape, and the effect they may have on nearby lung structures. This analysis helps determine the significance of the detected bullae in relation to COPD.

To improve the accuracy of the detection, the system cross-references the clinical data entered by the clinician with the detected imaging features. For example, if the patient has a history of smoking, the system factors this in as a potential risk factor for COPD. As a result, even smaller bullae may be flagged for closer inspection if the patient's clinical history suggests a higher risk profile. By mapping imaging features to clinical patterns, the system enhances its ability to differentiate between bullae that are high-risk and those that are incidental, thereby improving its diagnostic precision.

The system proceeds by integrating the visual data from the X-ray with the clinical information to generate a personalized risk score for each detected bullae. This score considers various factors, such as the bullae's size, location, the surrounding lung compression, and the patient's specific risk factors. This multimodal analysis leads to a more accurate risk assessment for COPD.

Following the analysis, an explainable report is generated, detailing each detected bullae's characteristics and the corresponding COPD risk score. The report includes clear explanations for the AI's assessment, citing specific imaging findings such as large bullae size or upper lobe involvement and clinical indicators, like a history of smoking or family history of lung disease. This transparency ensures that clinicians can understand and validate the AI's conclusions.

Finally, the clinician reviews the AI-generated report, using the insights to determine the next steps in the patient's care. This could include ordering follow-up imaging, recommending spirometry tests, or initiating patient monitoring. By providing an explainable, patient-specific report, the AI assists clinicians in making informed, personalized treatment decisions, ultimately improving early COPD detection and enabling better disease management.

Let us delve into a detailed example of the present disclosure.

Imagine a scenario where a healthcare professional is using the disclosed system to diagnose a patient with suspected COPD. The system receives a combination of X-ray images and clinical information, such as the patient's medical history, symptoms, and risk factors. The healthcare professional provides the patient's X-ray image along with details like smoking history, shortness of breath, and any other relevant clinical data. The system processes this information and performs the necessary steps to classify the COPD.

### Scenario 1:

In the case of a patient with emphysema, the system processes the X-ray images and clinical information. The X-ray image reveals hyperinflated lungs, a hallmark feature of emphysema. The system receives the X-ray image and clinical details such as smoking history and shortness of breath. The system first segments the lung regions, identifying areas of hyperinflation (such as the upper lobes). The system then analyses low-level features, like the texture of lung tissues, and high-level features, such as the curvature of the diaphragm. Based on these lung features and patterns, the system classifies the region of interest (ROI) into the “Emphysema” category, assigning a high confidence score. The system then generates the report that provides a classification of “Emphysema,” along with the personalized risk score, based on the patient's history and the clinical information. Additionally, the report includes an explanation of the findings, detailing the presence of hyperinflation and other characteristics of emphysema.

### Scenario 2:

For a patient with chronic bronchitis, the system analyses both the clinical information and the X-ray images. The clinical history includes a chronic cough and large amounts of thick sputum. The X-ray shows prominent bronchial markings, indicative of chronic bronchitis. The system processes the clinical information and X-ray images, segments the lung regions, and identifies areas with thickened bronchial walls. The segmentation model analyses the segmented lung regions, extracting features such as bronchial wall texture and position, and detecting patterns associated with chronic bronchitis. Based on this information, the system classifies the region of interest (ROI) into the “Chronic Bronchitis” category, assigning that particular ROI a high confidence score. The system then provides the report that includes the COPD classification of “Chronic Bronchitis,” the personalized risk score, and the explanation of the findings based on the X-ray image and the clinical information provided by the patient.

### Scenario 3:

In a case where the patient shows no significant signs of COPD, the system processes both the X-ray image and the clinical information. The X-ray may be normal, showing no hyperinflation, bronchial thickening, or other features associated with the COPD. The clinical history does not indicate any significant risk factors like smoking or chronic cough. The system receives the X-ray image and the clinical information, segments the lung regions, and determines that no significant regions of interest (ROIs) related to the COPD are present. The segmentation model analyses the X-ray image and finds no patterns indicative of the COPD. Based on this analysis, the system classifies the ROI into the “None” category with the high confidence score. Further, the system generates the report indicating “None” as the COPD category and provides the personalized risk score reflecting a low likelihood of the COPD condition. The explanation of findings confirms that there are no abnormal disease features in both the X-ray image and the clinical information.

In all the above examples, the system utilizes a combination of the segmentation model, the classification model, deep learning techniques, clinical information, and the personalized risk score to categorize the COPD and provide the detailed diagnostic report to various stakeholders.

A person skilled in the art will understand that the scope of the disclosure is not limited to scenarios based on the aforementioned factors and using the aforementioned techniques and that the examples provided do not limit the scope of the disclosure.

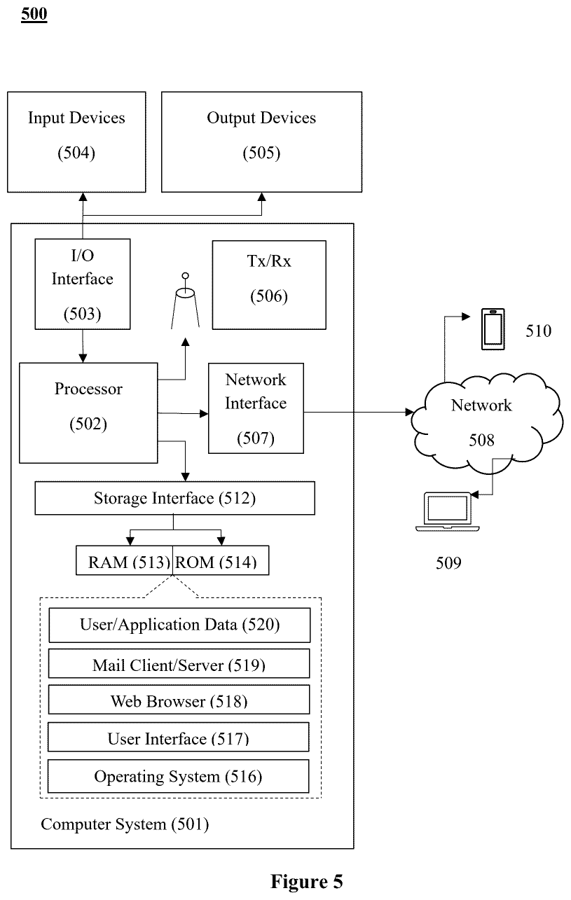

Now referring to FIG. 5 illustrates a block diagram (500) of an exemplary computer system (501) for implementing embodiments consistent with the present disclosure. Variations of computer system (501) may be used as the method for classification of the COPD. The computer system (501) may comprise a central processing unit (“CPU” or “processor”) (502). The processor (502) may comprise at least one data processor for executing program components for executing the user or the system generated requests. The user may include a person, a person using a device such as those included in this disclosure, or such a device itself. Additionally, the processor (502) may include specialized processing units such as integrated system (bus) controllers, memory management control units, floating point units, graphics processing units, digital signal processing units, or the like. In various implementations the processor (502) may include a microprocessor, such as AMD Athlon, Duron or Opteron, ARM's application, embedded or secure processors, IBM PowerPC, Intel's Core, Itanium, Xeon, Celeron or other line of processors, for example. Accordingly, the processor (502) may be implemented using mainframe, distributed processor, multi-core, parallel, grid, or other architectures. Some embodiments may utilize embedded technologies like application-specific integrated circuits (ASICs), digital signal processors (DSPs), or Field Programmable Gate Arrays (FPGAs), for example.

Processor (502) may be disposed in communication with one or more input/output (I/O) devices via I/O interface (503). Accordingly, the I/O interface (503) may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMAX, or the like, for example.

Using the I/O interface (503), the computer system (501) may communicate with one or more I/O devices. For example, the input device (504) may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, or visors, for example. Likewise, an output device (505) may be a user's smartphone, tablet, cell phone, laptop, printer, fax machine, video display (e.g., cathode ray tube (CRT), liquid crystal display (LCD), light-emitting diode (LED), plasma, or the like), or audio speaker, for example. In some embodiments, a transceiver (506) may be disposed in connection with the processor (502). The transceiver (506) may facilitate various types of wireless transmission or reception. For example, the transceiver (506) may include an antenna operatively connected to a transceiver chip (example devices include the Texas Instruments® WiLink WL1283, Broadcom® BCM4750IUB8, Infineon Technologies® X-Gold 618-PMB9800, or the like), providing IEEE 802.11a/b/g/n, Bluetooth, FM, global positioning system (GPS), and/or 2G/3G/5G/6G HSDPA/HSUPA communications, for example.

In some embodiments, the processor (502) may be disposed in communication with a communication network (508) via a network interface (507). The network interface (507) is adapted to communicate with the communication network (508). The network interface, coupled to the processor may be configured to facilitate communication between the system and one or more external devices or networks. The network interface (507) may employ connection protocols including, without limitation, direct connect, Ethernet (e.g., twisted pair 10/100/1000 Base T), transmission control protocol/internet protocol (TCP/IP), token ring, or IEEE 802.11a/b/g/n/x, for example. The communication network (508) may include, without limitation, a direct interconnection, local area network (LAN), wide area network (WAN), wireless network (e.g., using Wireless Application Protocol), or the Internet, for example. Using the network interface (507) and the communication network (508), the computer system (501) may communicate with devices such as shown as a laptop (509) or a mobile/cellular phone (510). Other exemplary devices may include, without limitation, personal computer(s), server(s), fax machines, printers, scanners, various mobile devices such as cellular telephones, smartphones (e.g., Apple iPhone, Blackberry, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, the computer system (501) may itself embody one or more of these devices.

In some embodiments, the processor (502) may be disposed in communication with one or more memory devices (e.g., RAM 513, ROM 514, etc.) via a storage interface (512). The storage interface (512) may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, or solid-state drives, for example.

The memory devices may store a collection of program or database components, including, without limitation, an operating system (516), user interface application (517), web browser (518), mail client/server (519), user/application data (520) (e.g., any data variables or data records discussed in this disclosure) for example. The operating system (516) may facilitate resource management and operation of the computer system (501). Examples of operating systems include, without limitation, Apple Macintosh OS X, UNIX, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like.

The user interface (517) is for facilitating the display, execution, interaction, manipulation, or operation of program components through textual or graphical facilities. For example, user interfaces may provide computer interaction interface elements on a display system operatively connected to the computer system (501), such as cursors, icons, check boxes, menus, scrollers, windows, or widgets, for example. Graphical user interfaces (GUIs) may be employed, including, without limitation, Apple Macintosh operating systems'Aqua, IBM OS/2, Microsoft Windows (e.g., Aero, Metro, etc.), Unix X-Windows, or web interface libraries (e.g., ActiveX, Java, JavaScript, AJAX, HTML, Adobe Flash, etc.), for example.

In some embodiments, the computer system (501) may implement a web browser (518) stored program component. The web browser (518) may be a hypertext viewing application, such as Microsoft Internet Explorer, Google Chrome, Mozilla Firefox, Apple Safari, or Microsoft Edge, for example. Secure web browsing may be provided using HTTPS (secure hypertext transport protocol), secure sockets layer (SSL), Transport Layer Security (TLS), or the like. Web browsers may utilize facilities such as AJAX, DHTML, Adobe Flash, JavaScript, Java, or application programming interfaces (APIs), for example. In some embodiments the computer system (501) may implement a mail client/server (519) stored program component. The mail server (519) may be an Internet mail server such as Microsoft Exchange, or the like. The mail server may utilize facilities such as ASP, ActiveX, ANSI C++/C#, Microsoft .NET, CGI scripts, Java, JavaScript, PERL, PHP, Python, or WebObjects, for example. The mail server (519) may utilize communication protocols such as internet message access protocol (IMAP), messaging application programming interface (MAPI), Microsoft Exchange, post office protocol (POP), simple mail transfer protocol (SMTP), or the like. In some embodiments, the computer system (501) may implement a mail client (520) stored program component. The mail client (520) may be a mail viewing application, such as Apple Mail, Microsoft Entourage, Microsoft Outlook, or Mozilla Thunderbird.

In some embodiments, the computer system (501) may store user/application data (521), such as the data, variables, records, or the like as described in this disclosure. Such databases may be implemented as fault-tolerant, relational, scalable, secure databases such as Oracle or Sybase, for example. Alternatively, such databases may be implemented using standardized data structures, such as an array, hash, linked list, struct, structured text file (e.g., XML), table, or as object-oriented databases (e.g., using ObjectStore, Poet, Zope, etc.). Such databases may be consolidated or distributed, sometimes among the various computer systems discussed above in this disclosure. It is to be understood that the structure and operation of the any computer or database component may be combined, consolidated, or distributed in any working combination.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

Various embodiments of the disclosure provide a non-transitory computer readable medium and/or storage medium, and/or a non-transitory machine-readable medium and/or storage medium having stored thereon, a machine code and/or a computer program having at least one code section executable by a machine and/or a computer for classification of the COPD. The at least one code section in the application server (101) causes the machine and/or computer including one or more processors to perform the steps, which includes receiving (301) the one or more inputs. In an embodiment, the one or more inputs may include at least one of the one or more X-ray images, the clinical information, associated with the user. Further, the steps may involve segmenting (302) the one or more lung regions from the one or more anatomical structures of the one or more X-ray images.

Furthermore, the steps may involve identifying (303) the one or more regions of interest (ROIs) from the segmented one or more lung regions. In an embodiment, the one or more ROIs are indicative of the COPD within the one or more lung regions. Furthermore, the steps may involve classifying (304) each of the one or more ROIs into the one or more COPD categories. Additionally, the steps may involve providing (305) the report indicating the one or more COPD categories.

Various embodiments of the disclosure encompass numerous advantages including the system and the method for classification of the COPD. The disclosed method and system have several technical advantages, but not limited to the following:
Enhanced Improved COPD Severity Assessment: The method leverages vector embeddings to capture contextual response, enabling precise identification of user intent. By classifying queries into transactional, non-domain specific, and non-transactional domain-specific categories, the method ensures accurate and efficient handling of diverse user inputs.
Integration of the Clinical Information: The inclusion of the clinical information alongside X-ray imaging enhances contextual understanding, leading to more informed and comprehensive COPD classification.
Cost-Effective and Scalable Solution: Compared to CT-based COPD detection methods, this approach is more affordable and accessible, making it feasible for large-scale population screening, especially in resource-limited settings.
Reduction in Radiation Exposure: Since the system relies on X-ray imaging instead of high-radiation CT scans, the system minimizes patient radiation exposure, making it suitable for repeated assessments and long-term monitoring.
Improved Lung Region Segmentation: The precise segmentation of lung regions from X-ray images ensures that only relevant anatomical structures are analyzed, improving the reliability of COPD classification.
Automated Report Generation: The system automatically generates reports summarizing COPD classification results, streamlining documentation and aiding healthcare providers in efficient patient management.

In summary, the technical advantages of this invention address the challenges associated with conventional systems such as the limitations of CT scans and X-ray images. While CT scans provide detailed lung assessments, their high cost, limited accessibility, and higher radiation exposure restrict their widespread use, particularly for routine screening. In contrast, X-rays, though providing comparatively lesser resolution as 2D images, offer significant advantages due to their accessibility, affordability, and ability to screen large populations. By integrating the one or more deep learning techniques with X-ray images and the clinical information, the system compensates for the resolution limitations of X-rays, allowing for the identification of subtle COPD features like lung hyperinflation, flattened diaphragms, and reduced vascular markings. Additionally, the AI-based techniques approach enables context-aware assessments by incorporating the clinical data, enhancing diagnostic accuracy and enabling early detection, even in resource-limited environments. The disclosed system makes X-rays a highly effective tool for initial COPD screening, categorization, and personalized assessments, offering a more accessible and cost-effective alternative to CT scans, particularly for widespread COPD detection and classification.

The claimed invention of the system and the method for classification of COPD involves tangible components, processes, and functionalities that interact to achieve specific technical outcomes. The system integrates various elements such as processors, memory, databases, matchmaking, offer calculation and informed displaying techniques to effectively perform the classification of COPD.

Furthermore, the invention involves a non-trivial combination of technologies and methodologies that provide a technical solution for a technical problem. While individual components like processors, databases, encryption, authorization and authentication are well-known in the field of computer science, their integration into a comprehensive system for classification of COPD brings about an improvement and technical advancement in the field of medical image processing and other related environments.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted for carrying out the methods described herein may be suited. A combination of hardware and software may be a general-purpose computer system with a computer program that, when loaded and executed, may control the computer system such that the computer system carries out the methods described herein. The present disclosure may be realized in hardware that includes a portion of an integrated circuit that also performs other functions.

A person with ordinary skills in the art will appreciate that the systems, modules, and sub-modules have been illustrated and explained to serve as examples and should not be considered limiting in any manner. It will be further appreciated that the variants of the above disclosed system elements, modules, and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules, and are not limited to any particular computer hardware, software, middleware, firmware, microcode, and the like. The claims can encompass embodiments for hardware and software, or a combination thereof.

While the present disclosure has been described with reference to certain embodiments, it will be understood by those skilled in the art that various changes may be made, and equivalents may be substituted without departing from the scope of the present disclosure. In addition, many modifications may be made to adapt a particular situation or material to the teachings of the present disclosure without departing from its scope. Therefore, it is intended that the present disclosure is not limited to the particular embodiment disclosed, but that the present disclosure will include all embodiments falling within the scope of the appended claims.

We claim:

1. A method (300) for classification of chronic obstructive pulmonary disease (COPD), characterized in that, the method (300) comprises:
receiving (301), via a processor (201), one or more inputs, wherein the one or more inputs comprise at least one of one or more X-ray images, a clinical information, or a combination thereof, associated with a user;
segmenting (302), via the processor (201), one or more lung regions from one or more anatomical structures of the one or more X-ray images;
identifying (303), via the processor (201), one or more regions of interest (ROIs) from the segmented one or more lung regions, wherein the one or more ROIs are indicative of COPD within the one or more lung regions;
classifying (304), via the processor (201), each of the one or more ROIs into one or more COPD categories; and
providing (305), via the processor (201), a report indicating the one or more COPD categories.

2. The method (300) as claimed in claim 1, wherein the clinical information comprises at least one of user history, risk factor, symptoms, bullae characteristics, typical anatomical changes, age, smoking history, personalized risk profile, clinical guidelines, previous diagnosis notes, family history of lung disease, or a combination thereof.

3. The method (300) as claimed in claim 1, comprises preprocessing the one or more X-ray images, wherein the preprocessing corresponds to one of noise reduction, contrast adjustment, normalization, or a combination thereof, wherein the preprocessing of the one or more X-ray images is performed before segmenting (302) the one or more lung regions from the one or more X-ray images.

4. The method (300) as claimed in claim 1, wherein the segmenting (302) of the one or more lung regions from one or more anatomical structures is performed using one or more deep learning models, wherein the one or more deep learning models corresponds to one of a UNet or a UNet++, DeepLab, Fully Convolutional Network (FCN), SegNet, R2U-Net (Recurrent Residual U-Net), ResUNet (U-Net with ResNet backbone), Densely Connected U-Net (DC-UNet), Mask R-CNN, or a combination thereof.

5. The method (300) as claimed in claim 4, comprises training the one or more deep learning models by utilizing the one or more X-ray images to identify one or more pixels, one or more features, one or more patterns associated with the one or more anatomical structures, wherein segmenting (302) the one or more lung regions from the one or more anatomical structures is performed based on the one or more pixels, one or more features, and one or more patterns identified during the training.

6. The method (300) as claimed in claim 1, wherein identifying (303) the one or more ROIs is performed by processing the segmented one or more lung regions utilizing at least one of a region-based convolutional neural network (R-CNN) deep learning model, a transformer-based neural network model, or a combination thereof; wherein processing the segmented one or more lung regions is performed at different scales to extract one or more lung features and analyze one or more patterns, indicative of the COPD.

7. The method (300) as claimed in claim 6, wherein processing the segmented one or more lung regions comprises:
extracting the one or more lung features from the segmented one or more lung regions, wherein each of the one or more lung features is associated with a weightage;
analyzing one or more patterns from the one or more lung features; and
integrating the one or more lung features and the one or more patterns, indicative of the COPD, for identifying (303) the one or more ROIs, wherein each of the one or more ROIs is assigned with a classification score based on the weightage associated with the one or more features.

8. The method (300) as claimed in claim 6, wherein the one or more lung features comprises low-level features and high-level features; wherein the low-level features comprise at least one of edges, gradients, shapes, texture or a combination thereof; wherein the high-level features comprise at least one of thickness, curvature, relative position, variation in pixel intensity, or a combination thereof.

9. The method (300) as claimed in claim 6, wherein the one or more patterns comprises at least one of flattened diaphragm, thickened bronchial walls, hyperinflation, bronchial thickening, radiolucency, position of diaphragm, narrow heart, air bullae in upper lobes, edges of the diaphragm, edges of bronchial wall, texture and thickness of bronchial walls, hallmark of hyperinflated lungs.

10. The method (300) as claimed in claim 1, comprises processing the clinical information using one or more language models, wherein processing the clinical information comprises parsing a clinical text from the clinical information and encoding the clinical text into clinical embeddings; wherein the one or more language models corresponds to NLP, LLM, or a combination thereof.

11. The method (300) as claimed in claim 10, comprises integrating the one or more lung features with the clinical embeddings for identifying contextual insights about the COPD; wherein integration is performed using one or more multi-modal techniques; wherein the one or more multi-modal techniques comprises one of co-attention mechanism, cross attention mechanism, late fusion, or a combination thereof.

12. The method (300) as claimed in claim 1, wherein classifying (304) each of the one or more ROIs into one or more COPD categories is performed based on the one or more lung features, the one or more patterns, the clinical embeddings, or a combination thereof; wherein the one or more COPD categories comprise None, Emphysema and Chronic Bronchitis; wherein classifying (304) each of the one or more ROIs into the one or more COPD categories is performed utilizing at least one of logistic regression, SVM, feed forward network (MLP), transformer based classifier, or a combination thereof; wherein each of the one or more COPD categories is assigned with a confidence score, wherein a cumulative sum of each of the confidence score corresponding to the one or more COPD categories is equal to one.

13. The method (300) as claimed in claim 12, wherein classification (304) of each ROI from the one or more ROIs corresponds to a COPD category from the one or more COPD categories, with a highest confidence score among other COPD categories.

14. The method (300) as claimed in claim 2, comprises calculating a personalized risk score, corresponding to the user, based on the one or more COPD categories and the risk factor associated with the user; wherein the personalized risk score is indicative of an estimate of likelihood of the COPD.

15. The method (300) as claimed in claim 14, wherein the one or more COPD categories is provided over the one or more X-ray images in the report, wherein the report comprises the one or more COPD categories, personalized risk score and an explanation of findings.

16. A system (100) for classification of chronic obstructive pulmonary disease (COPD), characterized in that, the system (100) comprises:
a processor (201),
a memory (202) communicatively coupled with the processor (201), wherein the memory (202) is configured to store one or more executable instructions, which cause the processor (201) to:
receive (301) one or more inputs, wherein the one or more inputs comprise at least one of one or more X-ray images, a clinical information, or a combination thereof, associated with a user;
segment (302) one or more lung regions from one or more anatomical structures of the one or more X-ray images;
identify (303) one or more regions of interest (ROIs) from the segmented one or more lung regions, wherein the one or more ROIs are indicative of COPD within the one or more lung regions;
classify (304) each of the one or more ROIs into one or more COPD categories; and
provide (305) a report indicating the one or more COPD categories.

17. A non-transitory computer-readable storage medium having stored thereon, a set of computer-executable instructions causing a computer comprising one or more processors to perform steps comprising:
receiving (301) one or more inputs, wherein the one or more inputs comprise at least one of one or more X-ray images, a clinical information, or a combination thereof, associated with a user;
segmenting (302) one or more lung regions from one or more anatomical structures of the one or more X-ray images;
identifying (303) one or more regions of interest (ROIs) from the segmented one or more lung regions, wherein the one or more ROIs are indicative of COPD within the one or more lung regions;
classifying (304) each of the one or more ROIs into one or more COPD categories; and
providing (305) a report indicating the one or more COPD categories.
